(19) United States
(12) Patent Application Publication
(10) Pub. No.: US 2002/0188147 A1
(43) Pub. Date: Dec. 12, 2002

(54) Process of using 1-methyl-2-pyrrolidinone as a pyridine replacement

(76) Inventor: Allen D. Johnston, West Chester, PA (US)

Correspondence Address: CONNOLLY BOVE LODGE & HUTZ, LLP, 1220 N MARKET STREET, P O BOX 2207, WILMINGTON, DE, 19899

(21) Appl. No.: 10/121,888

(22) Filed: Apr. 12, 2002

Related U.S. Application Data
(60) Provisional application No. 60/297,196, filed on Jun. 8, 2001.

(51) Int. Cl. C 07C 3 9/28; C07C 69/76

(52) U.S. Cl. 558/56; 560/231

(57) ABSTRACT

A process to prepare an ester which reacts an acid and a base in a solvent which contains 1-methyl-2-pyrrolidinone. The use of 1-methyl-2-pyrrolidinone can replace pyridine solvent. The pyridine solvent is a challenge for disposal and safe handling.

### RELATED APPLICATIONS

This application claims benefit to Provisional application No. 60/297,196 filed Jun. 8, 2001 which is incorporated by reference in its entirety for all useful purposes.

### BACKGROUND OF THE INVENTION

The reaction between an acid chloride and an alcohol to prepare an ester is one of the classic and best methods available. Generally this reaction is called the Schotten-Baumann procedure. It usually employs an aqueous base to absorb the acid side product by the reaction. The reaction is run in pyridine solvent. The choice of solvent for the reaction is based upon solubilities of the reactants. Frequently the alcohols may be soluble in the aqueous layer, but the acid chlorides and the ester may not be. The pyridine allows for all the reactants to be soluble simultaneously and also absorbs the acid byproduct. The pyridine solvent is a challenge for disposal and safe handling. It is a flammable liquid and has a strong odor.

### SUMMARY OF THE INVENTION

An object of the invention was to find a non-flammable solvent that could be used in lieu of the pyridine solvent.

Another object of the invention was to find a safe solvent for handling and disposal that could be used in place of the known pyridine solvent.

The use of 1-methyl-2-pyrrolidinone (“NMP”) in place of pyridine reduces the could be used in place of the known pyridine solvent.

The use of 1-methyl-2-pyrrolidinone (NMP) in place of pyridine reduces the challenges. The NMP is listed as an irritant and is not flammable. The odor of NMP is mild. NMP delivers the same solubility parameters for the reaction that pyridine does.

### A DETAILED DESCRIPTION OF THE INVENTION

The use of NMP in place of pyridine has now been determined to be an excellent substitute. It meets the objectives as stated above. The NMP is an irritant that is not flammable and has a mild odor. NMP delivers the same solubility parameters for the reaction that pyridine does. Alcohols, acid chlorides and esters are soluble in NMP. A stoichiometric measure of acid absorbing base material is required for the esterification to proceed. A range of bases can be used for the reaction that can absorb the acid, preferably the acid is HX wherein X is any type of leaving group, preferably halogen, acetate, tosylate or mesylate. The bases are preferably soluble, such as but not limited to amine bases and more preferably tertiary amine bases, such as, but not limited to triethylamine or N,N-dimethylbenzylamine, or insoluble bases such as, but not limited to poly-4-vinyl pyridine, such as Reillex 402 or 420. The isolation of the ester products from the reaction mixture is the same or about the same for each solvent. The yields are the same or about the same.

### EXAMPLE

The esterification work that was performed was as follows:

Elvanol HV 17 g,

Elvanol 71-30 17 g,

NMP 700 mL,

N,N-dimethyl-benzylamine 200 mL,

Naphthoyl Chloride 81.88 g and

Cinnamoyl Chloride 132.91 g.

The alcohols were suspended in the NMP at 70° C. under a blanket of dry carbon dioxide gas. The atmospheric water was preferably removed from the reaction. It can also be accomplished with a dry inert gas such as, but not limited to, carbon dioxide, nitrogen or argon. Carbon dioxide was used in this example. The acid chlorides were added followed by the amine in 50 mL aliquots. The solution temperature rose to 82° C. with the first amine addition. The next 50 mL addition raised the reaction temperature to 100° C. The solution was cooled to 90° C. for the third addition of 50 mL of amine. The temperature rose to 115° C. The last addition of amine occurred when the solution had cooled to 100° C. The exotherm finally halted at 123 C and the solution was cooled to 70° C. The solution was diluted with methanol after cooling to 55° C. The solid was precipitated by adding small quantities to water. The results achieved using the NMP in place of pyridine were of a similar level. However, as stated above, the use of NMP avoided the problems of handling and disposal and the strong odor that are associated with pyridine. Again, this process can be practiced without any pyridine present. The reaction can take place with all or substantially all or even some of the pyridine being replaced with NMP.

While there is shown and described certain specific structures embodying the invention, it will be manifest to those skilled in the art that various modifications and rearrangements of the parts may be made without departing from the spirit and scope of the underlying inventive concept and that the same is not limited to the particular forms herein shown and described.

I claim:

1. A process to prepare an ester which comprises reacting an acid and a base in a solvent which comprises 1-methyl-2-pyrrolidinone.

2. The process as claimed in claim 1, wherein said base is an amine base or an insoluble base.

3. The process as claimed in claim 1, wherein said base is a tertiary amine.

4. The process as claimed in claim 1, wherein said base is triethylamine or N,N-dimethylbenzylamine.

5. The process as claimed in claim 1, wherein said base is poly-4-vinyl pyridine.

6. The process as claimed in claim 1, wherein said acid is HX wherein X is a leaving group.

7. The process as claimed in claim 1, wherein X is halogen, acetate, tosylate or mesylate.

8. The process as claimed in claim 2, wherein said acid is HX wherein X is chloride, acetate, tosylate or mesylate.

9. The process as claimed in claim 3, wherein said acid is HX wherein X is chloride, acetate, tosylate or mesylate.

10. The process as claimed in claim 4, wherein said acid is HX wherein X is chloride.

11. The process as claimed in claim 5, wherein said acid is HX wherein X is chloride, acetate, tosylate or mesylate.

12. The process as claimed in claim 1, wherein the process is carried out in the substantial or complete absence of pyridine.

13. The process as claimed in claim 2, wherein the process is carried out in the substantial or complete absence of pyridine.

14. The process as claimed in claim 4, wherein the process is carried out in the substantial or complete absence of pyridine.

15. The process as claimed in claim 5, wherein the process is carried out in the substantial or complete absence of pyridine.

16. The process as claimed in claim 7, wherein the process is carried out in the substantial or complete absence of pyridine.

17. The process as claimed in claim 8, wherein the process is carried out in the substantial or complete absence of pyridine.

18. The process as claimed in claim 9, wherein the process is carried out in the substantial or complete absence of pyridine.

19. The process as claimed in claim 10, wherein the process is carried out in the substantial or complete absence of pyridine.

20. The process as claimed in claim 11, wherein the process is carried out in the substantial or complete absence of pyridine.
